(19) United States
(12) Patent Application Publication
(10) Pub. No.: US 2008/0194810 A1
(43) Pub. Date: Aug. 14, 2008

(54) Method for Purifying Hyaluronic Acid Using Calcium Salt and Phosphate Salt, or Calcium Phosphate Salt

(75) Inventors: Tai-Hyo Kim, Chungcheongnam-do (KR); Hui-Lee Kim, Chungcheongnam-do (KR); Sun-Young Park, Chungcheongnam-do (KR); Dong-Kwang Jang, Gyeonggi-do (KR)

Correspondence Address: BIRCH STEWART KOLASCH & BIRCH, PO BOX 747, FALLS CHURCH, VA, 22040-0747, US

(73) Assignee: T & LIFE SYSTEM CO., LTD., Asan-si, Chungcheongnam-do (KR)

(21) Appl. No.: 11/629,819

(22) PCT Filed: Jun. 15, 2005
(86) PCT No.: PCT/KR05/01821

§ 371 Date: Jun. 27, 2007

(30) Foreign Application Priority Data
Jun. 16, 2004 (KR) 10-2004-0044450

(51) Int. Cl. C12P 19/12

(52) U.S. Cl. 536/123.13

(57) ABSTRACT

The present invention relates to a method for purifying hyaluronic acid using calcium salt and phosphate salt, or calcium phosphate salt, specifically to a method for purifying hyaluronic acid characterized by effectively removing lipids, nucleic acids and proteins from an extract or a culture solution which contains hyaluronic acid by treating with calcium salt and phosphate salt, or calcium phosphate salt. According to the method of the present invention, it is possible to purify hyaluronic acid more effectively, by avoiding a use of toxic organic materials as well as reducing additional processes.

### TECHNICAL FIELD

The present invention relates to a method for purifying hyaluronic acid using calcium salt and phosphate salt, or calcium phosphate salt, specifically to a method for purifying hyaluronic acid by effectively removing lipids, nucleic acids and proteins from an extract or a culture solution which contains hyaluronic acid by treating the extract or the culture solution with calcium salt and phosphate salt, or calcium phosphate salt.

### BACKGROUND ART

Hyaluronic acid is an acidic muco-polysaccharide that is one of glycosaminoglycans present in a living body, having a repetitive molecular structure comprising D-glucuronic acid and N-acetylglucosamine with a molecular weight of 1,000 Da-10 kDa, and having very high viscosity and elasticity.

Hyaluronic acid is present in all part of human body, and shows various functions at each part of the body. It is a biopolymer which has excellent water retaining property, good viscosity and elasticity, and functions as: a lubricant to make the body movement smooth, a media which manages the transfer of physiologically active materials and induces cell differentiation and growth by specific interactions with the cells, and in extracelluar matrix, a supporter for proteins and glycoproteins such as collagen, elastin, chondroitin sulfate and the like which support tissue. Owing to such properties as above-mentioned, hyaluronic acid is used as a therapeutic agent and a pain-relieving agent for age-related degenerative arthritis, as an ophthalmic operation supplementary material and a main ingredient of a medicament for xerophthalmia, and, in the limelight, as a supplementary material for plastic surgery such as for complementing wrinkles and depressed skin tissue.

Hyaluronic acid currently used in medical field, specifically for ophthalmic use, is generally contained quantitatively 90% or more in products on a dry basis, and has an average molecular weight of 1.9×0⁶-3.9×0⁶ g/mol and an average degree of polymerization (n) of 4,700-9,600. For an injection solution for arthritis, products having various ranges of molecular weight distribution are currently available in the market. Since the hyaluronic acid for medical use is used for a drug for injection, the regulations regarding its safety are very strict such that it requires the degree of purity which does not even allow incorporation of proteins or other acidic mucopolysaccharides such as chondroitin sulfate, heparin sulfate, dermatan sulfate and the like.

The official standard for hyaluronic acid for medical (ophthalmic) use issued by Korea Food and Drug Administration is represented in the following Table 1.

TABLE 1
Official standard for hyaluronic acid for medical (ophthalmic) use

| Content | Test method | standard |
|---|---|---|
| Hyaluronic acid | Carbazole method (Anal. Biochem. 4, | >90% |
| | 330 (1962)) | |
| Proteins | Lowry's method | <0.02% |
| Nucleic acids | Absorbance at 260 nm wavelength | N.D.* |
| Other acidic mu- | Electrophoresis (Japanese Journal of | N.D.* |
| copolysaccharides | Bio-chemistry 38, 479 (1966)) | |
| viscosity | Intrinsic viscosity | 30-55 dL/g |

*N.D.: not detected

Hyaluronic acid for medical use may be obtained by extraction from animal tissue or recovering from a fermented culture of microorganism, and it is reported that the hyaluronic acid extracted from animal tissue has a better effect than that from microbial culture (Journal of the European Academy of Dermatology and Venereology, 13, 183-192, (1999)). Therefore, various methods for isolating and purifying hyaluronic acid from animal tissue, specifically from rooster combs are disclosed.

U.S. Pat. No. 4,141,973 discloses a method for isolating and purifying hyaluronic acid from rooster combs using chloroform through several times of extraction processes.

EP No. 01238572 discloses a method for isolating and purifying hyaluronic acid having low molecular weight from rooster combs by using an ultrafiltration membrane according to the molecular weight.

WO 9200861 discloses a method for obtaining hyaluronic acid by treating the extract from rooster combs with protease, converting the resultant into quaternary ammonium salts, removing the ammonium salts with an organic solvent, subjecting the resultant to an ion exchange resin(Dowex) to obtain fractions, then precipitating the hyaluronic acid fraction by the treatment with cetylpyridinium chloride and adding a water-soluble organic solvent to the precipitated hyaluronic acid fraction to precipitate hyaluronic acid.

Additionally, Korean laid-open patent publication No. 1987-0008904 discloses a method for producing hyaluronic acid which comprises treating the extract from rooster combs with a protease, removing lipids and proteins therefrom with an organic solvent such as butanol and the like, carrying out sterilizing and precipitating simultaneously with cetylpyridinium chloride to obtain fractions, adding thereto a water-soluble organic solvent to precipitate hyaluronic acid.

### DISCLOSURE OF INVENTION

### Technical Problem

However, the above-listed methods have problems of applying them to the mass production scale such as complicated process, low production yield, massive cost required for purification and water treatment owing to the use of organic solvents, and autolysis of the resulted hyaluronic acid which can be occurred during the long-time multistep processes. Further, these methods have another significant problem of the use of toxic organic solvents, which are not allowable for producing products for medical use.

### Technical Solution

The present invention is to solve the problems of convention techniques as described above. Therefore, the objects of the invention is to provide a method for purifying hyaluronic acid in more effective way, by excluding the use of toxic organic materials and reducing the number of steps required for the process with respect to the conventional hyaluronic acid purification process.

According to the present invention, provided is a method for purifying hyaluronic acid comprising the steps of: (1) treating a crude extract of hyaluronic acid with calcium salt and phosphate salt, or calcium phosphate salt, wherein the crude extract is obtained by treating natural product containing hyaluronic acid with protease, or from a culture solution containing hyaluronic acid originated from microorganism; and (2) purifying the hyaluronic acid by filtering the resultant from the step (1) and precipitating the hyaluronic acid from the filtrate.

### Mode for the Invention

In the method of the present invention, the crude extract used in the step (1) is obtained by treating natural product containing hyaluronic acid with protease, or from a culture solution containing hyaluronic acid originated from microorganism which can produce hyaluronic acid. The natural product containing hyaluronic acid is not specifically limited as long as it can provide purified hyaluronic acid according to the present invention, and rooster combs are mainly used. As for the treatment condition, it is not specifically restricted as long as the treatment with a protease can be available, and conventionally known treatment methods using protease may be used. As for the microorganism which can produce hyaluronic acid, conventionally known microorganism for the production of hyaluronic acid may be used, and a microorganism culture solution containing hyaluronic acid obtained therefrom by using any known method can also be used without any limitation in the purifying method of the present invention.

Hereinafter, the process for obtaining a crude extract of hyaluronic acid is further illustrated in detail by treating the natural product containing hyaluronic acid with protease through one preferred embodiment of the present invention.

According to one preferred embodiment of the present invention, for obtaining the crude extract of hyaluronic acid by treating a natural product containing hyaluronic acid with protease, firstly rooster combs as a natural product containing hyaluronic acid are finely ground and washed with ethanol. Next, the ground and washed rooster combs are added with a suitable amount of distilled water, then stirred in the presence of protease, and allowed to stand at 30-60° C. for 3-72 hours. As a result of standing, phase separation is occurred, then the separated upper phase comprising lipids is discarded, and the extract solution of the lower phase is taken and filtered. The extract obtained from the filtration is heated to 30-60° C., and then treated again with protease added thereto for 3-24 hours. After the second treatment, the resulted extract is filtered to give a hyaluronic acid extract. This hyaluronic acid extract may be used as a crude extract of hyaluronic acid in the step (1) without any further treatment. However, as the crude extract of hyaluronic acid, it is preferred to use a hyaluronic acid powder obtained by adding NaCl, in a quantified amount to provide the NaCl concentration of 0.3-1M, to the above-obtained hyaluronic acid extract, precipitating hyaluronic acid in 95% ethanol, filtering the solution to obtain hyaluronic acid precipitates, and lyophilizing or vacuum drying the hyaluronic acid precipitates. The hyaluronic acid powder thus obtained is dissolved into distilled water to prepare an aqueous solution of hyaluronic acid at an appropriate concentration for use.

According to the method for purifying hyaluronic acid of the present invention, in the step (1), the crude extract of hyaluronic acid is treated with calcium salt and phosphate salt, or calcium phosphate salt to remove lipids, nucleic acids and proteins from the crude extract. Particularly, in the step (1), it is preferred that the crude extract of hyaluronic acid is treated at the temperature range of 4-90° C. with calcium salt and phosphate salt, or calcium phosphate salt.

In the step (1), the useful calcium salt may be at least one selected from the group consisting of Ca(OH)₂, CaCO₃, Ca(NO₃)₂.4H₂O, Ca(CH₃COO)₂.H₂O, CaCl₂ and CaSO₄.2H₂O, and the useful phosphate salt may be at least one selected from the group consisting of H₃PO₄, (NH₄)₂HPO₄, H₄P₂O₇, NaH₂PO₄ and KH₂PO₄, but those useful salts are not restricted to the above-listed compounds. The molar ratio of the calcium salt to the phosphate salt, Ca/P, is preferably 0.5-3.0, in view of the efficiency of purifying hyaluronic acid.

Further, in the step (1), the calcium phosphate salt may be at least one selected from the group consisting of compounds represented in the following Table 2.

TABLE 2

| Formula | Name | Abb. |
|---|---|---|
| Ca₄(PO₄)₂ | Tetracalcium phosphate (Hilgenstockite) | TeCP(TTCP) |
| Ca₁₀(PO₄)₆(OH)₂ | Hydroxyapatite | HA(HAp) |
| Ca₁₀₋ₓ(HPO₄)ₓ(PO₄)₆₋ₓ(OH)₂₋ₓ•nH₂O | onstoichiometric hydroxyapatite | HA(HAp) |
| [0 < x ≦ 1, 0 < n ≦ 2] | | |
| Ca₃(PO₄)₂•nH₂O [n = 3-4.5] | morphous calcium phosphate | ACP |
| Ca₃(PO₄)₂ | Tricalcium phosphate (α, βWhitlockite) | TCP |
| Ca₈H₂(PO₄)₆•5H₂O | Octacalcium phosphate | CP |
| CaHPO₄•2H₂O | Dicalcium phosphate dihydrate (Burcite) | CPD |
| CaHPO₄ | Dicalcium phosphate anhydrate | DCPA |
| | (Monetite) | |
| Ca₂P₂O₇ | Calcium pyrophosphate | CPP |
| Ca₂P₂O₇•2H₂O | Calcium pyrophosphate dihydrate | CPPD |
| Ca₇(P₅O₁₆)₂ | Heptacalcium phosphate (Tromelite) | HCP |
| Ca₄H₂P₆O₂₀ | Tetracalcium dihydrogen phosphate | TDCP |
| Ca(H₂PO₄)₂•H₂O | Monocalcium phosphate monohydrate | MCPM |
| Ca(PO₃)₂ | Calcium metaphosphate | CMP |

In the step (1), the crude extract of hyaluronic acid is treated in the form of an aqueous solution, and it is preferred that the calcium salt and the phosphate salt, or the calcium phosphate salt is mixed, in the form of aqueous solution, with the aqueous solution of the crude extract of hyaluronic acid.

The treatment of the aqueous solution of the crude extract of hyaluronic acid with the aqueous solution of the salts as above, is carried out under conditions of pH 5-14, and preferably pH 8-10. When the pH is lower than 5 during the treatment, precipitation of impurities is not effectively achieved, and as a result, the purification of hyaluronic acid becomes insufficient. The aqueous solution of the crude extract of hyaluronic acid is treated under the condition of pH 5-14, simultaneously with mixing or after mixing. The expression “simultaneously with mixing” means that the pH of the aqueous solution of calcium salt and phosphate salt, or calcium phosphate salt, is pre-conditioned to the range of 5-14, and then the aqueous solution of the crude extract of hyaluronic acid is mixed with the pH-conditioned aqueous solution so as to provide the conditions of pH 5-14 to the aqueous solution of the crude extract of hyaluronic acid as being mixed. The expression “after mixing” means that, after mixing the aqueous solution of calcium salt and phosphate salt, or calcium phosphate salt, with the aqueous solution of the crude extract of hyaluronic acid, the pH of the mixed solution is adjusted to 5-14 so as to let the aqueous solution of the crude extract of hyaluronic acid be in the condition of pH 5-14 after mixing with the aqueous solution of salts.

In the above, for bring the pH condition in the range of 5-14, basic compounds may be used. As for the basic compound, it is preferred to use at least one selected from the group consisting of NaOH, KOH, LiOH, NH₄ OH, Mg(OH)₂, trishydroxymethyl-aminomethane, bis-2-hydroxyethyl-aminotris-hydroxymethyl-methane and 1,3-bis-[tris-hydroxymethyl]-methyl-aminopropane, however it is not restricted by the above mentioned compounds.

When treating the aqueous solution of the crude extract of hyaluronic acid with the aqueous solution of salts, an acidic aqueous solution which contains inorganic acid, organic acid or a mixture thereof may be used. As for the acidic aqueous solution, it is preferred to use an acidic aqueous solution prepared by using at least one acidic compound selected from the group consisting of HCl, HNO₃, HF, H₂SO₄, HClO₄, HClO, H₂CO₃, CH₃COOH, C₂H₂(COOH)₂, HOOCCH₂CH₂COOH, CHCOCOOH and CH₃(CH₂)₁₄COOH, however useful inorganic or organic acids are not limited to those above-mentioned.

In the step (1), the concentration of the aqueous solution of phosphate salt, calcium salt and calcium phosphate salt, the mixing ratio and mixing order of the aqueous solution of the crude extract of hyaluronic acid and aqueous solutions of the salts, the concentration of the basic compound for controlling pH, the concentration of the acidic aqueous solution containing inorganic acid, organic acid or a mixture thereof and the like may be varied by specific treatment process on each case. Therefore, the treatment method of the step (1) is, hereinafter, further illustrated through the preferred embodiments given below.

According to the preferred embodiment of the treatment method in the step (1), the treatment method of the crude extract of hyaluronic acid may be largely divided in two methods, one using calcium salt and phosphate salt respectively, and the other using calcium phosphate salt.

Firstly, in the method using calcium salt and phosphate salt respectively, an aqueous solution (first aqueous solution) which does not contain phosphate ions but calcium ions is prepared with Ca(OH)₂, CaCO₃, Ca(NO₃)₂.4H₂O, Ca(CH₃COO)₂.H₂O, CaCl₂, CaSO₄.2H₂O and the like, and an aqueous solution (second aqueous solution) which does not contain calcium ions but phosphate ions is prepared with H₃PO₄, (NH₄)₂HPO₄, H₄P₂O₇, NaH₂PO₄, KH₂PO₄ and the like, respectively. The calcium ion concentration of the first aqueous solution and the phosphate ion concentration of the second aqueous solution are suitably 0.001-10M, preferably 0.01-2M, and the first and the second aqueous solution are used to provide the molar ratio of the calcium salt to the phosphate salt, Ca/P, preferably of 0.5-3.0, and more preferably of 1.0-2.0.

The treatment method of the crude extract of hyaluronic acid with the first and the second aqueous solution as prepared above may be various by the order of treating procedures as the following methods.

In a first method, 0.05-1% concentration of an aqueous solution of the crude extract of hyaluronic acid is added to the first aqueous solution at the amount which can obtain a mixed solution having the mixing ratio of the first aqueous solution/the aqueous solution of the crude extract of hyaluronic acid being 0.001-100 by volume, preferably 0.1-2 by volume, and stirred for about 10 minutes to 3 hours, preferably for 30 minutes to 1 hour for sufficient mixing. Subsequently, the second aqueous solution is added to the mixed solution of the first aqueous solution and the aqueous solution of the crude extract of hyaluronic acid at the amount which can obtain the molar ratio of Ca/P in the mixed solution of the first and the second aqueous solution of 0.5-3.0. To the mixed aqueous solution of the first aqueous solution, the second aqueous solution and the aqueous solution of the crude extract of hyaluronic acid thus obtained, an aqueous solution of at least one basic compound selected from the group consisting of NaOH, KOH, LiOH, NH₄OH, Mg(OH)₂, trishydroxymethyl-aminomethane, bis-2-hydroxyethyl-aminotris-hydroxymethyl-methane and 1,3-bis-[tris-hydroxymethyl]-methyl-aminopropane is added at the amount which can provide the pH in the range of 5-14, preferably of 8-10, and stirred for about 30 minutes-24 hours, preferably 1 hour-3 hours.

In a second method, the aqueous solution of the crude extract of hyaluronic acid is added to the second aqueous solution at the amount which can obtain a mixed solution having the mixing ratio of the second aqueous solution/the aqueous solution of the crude extract of hyaluronic acid being 0.001-100 by volume, preferably 0.1-2 by volume, and stirred for about 10 minutes to 3 hours, preferably for 30 minutes to 1 hour for sufficient mixing. Subsequently, the first aqueous solution is added to the mixed solution of the second aqueous solution and the aqueous solution of the crude extract of hyaluronic acid at the amount which can obtain the molar ratio of Ca/P in the mixed solution of the first and the second aqueous solution of 0.5-3.0, though it may varied to the small extent by the concentration. To the mixed aqueous solution of the first aqueous solution, the second aqueous solution and the aqueous solution of the crude extract of hyaluronic acid thus obtained, the aqueous solution of the basic compound as described above is added at the amount which can provide the pH in the range of 5-14, preferably of 8-10, and stirred for about 30 minutes-24 hours, preferably 1 hour-3 hours.

In a third method, the aqueous solution of the basic compound is added to the first and the second aqueous solution, respectively, to bring the pH of each solution in the range of 5-14, preferably 8-10. The resulted pH-controlled first aqueous solution and the aqueous solution of the crude extract of hyaluronic acid are mixed together at the amount which can obtain the mixing ratio of the first aqueous solution/the aqueous solution of the crude extract of hyaluronic acid of 0.001-100 by volume and preferably 0.1-2 by volume, and subsequently, the second aqueous solution is added thereto at the amount which can obtain the molar ratio of Ca/P in the mixed solution of the first and the second aqueous solution of 0.5-3.0. Alternatively, the pH-controlled second aqueous solution and the aqueous solution of the crude extract of hyaluronic acid are mixed together at the amount which can obtain the mixing ratio of the second aqueous solution/the aqueous solution of the crude extract of hyaluronic acid of 0.001-100 by volume, preferably 0.1-2 by volume, and subsequently, the first aqueous solution is added thereto at the amount which can obtain the molar ratio of Ca/P in the mixed solution of the first and the second aqueous solution of 0.5-3.0. The mixed aqueous solution of the first aqueous solution, the second aqueous solution and the aqueous solution of the crude extract of hyaluronic acid thus obtained are stirred for about 30 minutes-24 hours, preferably 1 hour-3 hours.

In another alternative method, the aqueous solution of the crude extract of hyaluronic acid is added to the first and the second aqueous solution, respectively, and the pH condition of the each solution is controlled to 5-14, and preferably to 8-10 with the aqueous solution of the basic compound. Then, the mixed aqueous solution of the first aqueous solution and the aqueous solution of the crude extract of hyaluronic acid and the mixed aqueous solution of the second aqueous solution and the aqueous solution of the crude extract of hyaluronic acid can be mixed.

In a fourth method, the aqueous solution of the basic compound is added to the mi xed aqueous solution of the first and the second aqueous solution, which has the molar ratio of Ca/P of 0.5-3.0, to bring the pH in the range of 5-14, and preferably 8-10, and stirred for 10 minutes to 24 hours. The mixed aqueous solution of the first aqueous solution and the second aqueous solution thus obtained is mixed with the aqueous solution of the crude extract of hyaluronic acid at the amount which can obtain the mixing ratio of the first aqueous solution/the aqueous solution of the crude extract of hyaluronic acid of 0.001-100 by volume, preferably of 0.1-2 by volume, and stirred for about 30 minutes to 24 hours, preferably 1 hour to 3 hours.

In the method using calcium salt and phosphate salt respectively, the acidic aqueous solution of inorganic acid, organic acid or a mixture thereof may be used as follows.

In a first method among methods using an acidic aqueous solution of inorganic acid, organic acid or a mixture thereof, an acidic aqueous solution is prepared by using at least one selected from the group consisting of HCl, HNO₃, HF, H₂SO₄, HClO₄, HClO, H₂CO₃, CH₃COOH, C₂H₂(COOH)₂, HOOCCH₂CH₂COOH, CHCOCOOH and CH₃(CH₂)₁₄COOH to provide the concentration of 0.01-10M, preferably 0.1-2M, and calcium ions are added thereto. The calcium ion concentration in the acidic aqueous solution is suitably 0.001-10M, and preferably 0.01-2M. The calcium ions can be applied thereto by adding the first aqueous solution prepared as above, or by dissolving the calcium salt for the preparation of the first aqueous solution directly into the acidic aqueous solution.

The acidic aqueous solution which contains calcium ions thus prepared is mixed with the aqueous solution of the crude extract of hyaluronic acid at the amount which can obtain the mixing ratio of the acidic aqueous solution containing calcium ions/the aqueous solution of the crude extract of hyaluronic acid of 0.001-100 by volume, preferably of 0.1-2 by volume, and stirred for about 10 minutes to 5 hours, preferably for 30 minutes to 1 hour. Thus prepared mixed aqueous solution of the calcium ion-containing acidic aqueous solution and the aqueous solution of the crude extract of hyaluronic acid is further mixed with the second aqueous solution at the amount which can obtain the molar ratio of Ca/P of 0.5-3.0, and sufficiently stirred. Alternatively, it is possible to mix the aqueous solution of the crude extract of hyaluronic acid with the second aqueous solution, and then with the calcium ion-containing acidic aqueous solution.

The pH of the mixed aqueous solution of the calcium ion-containing acidic aqueous solution, the second aqueous solution and the aqueous solution of the crude extract of hyaluronic acid as prepared above, is controlled to be in the range of 5-14, preferably of 8-10 with the aqueous solution of the basic compound, and stirred for 30 minutes to 24 hours, preferably for 1-3 hours.

As an alternative, before mixing the calcium ion-containing acidic aqueous solution with the second aqueous solution, the pH of each solution may be previously controlled to be in the range of 5-14 with the aqueous solution of the basic compound, respectively. Still as another alternative, it is also possible that the calcium ion-containing acidic aqueous solution and the second aqueous solution are mixed together, then pH of the resulted mixed solution is controlled to be in the range of 5-14 with the aqueous solution of the basic compound, and the aqueous solution of the crude extract of hyaluronic acid is added to the pH-controlled mixed solution.

In a second method among methods using the acidic aqueous solution of inorganic acid, organic acid or a mixture thereof, phosphate ions are added to the acidic aqueous solution prepared as above. The concentration of the phosphate ions in the acidic aqueous solution is suitably 0.001-10M, and preferably 0.01-2M. The phosphate ions can be applied by adding the second aqueous solution prepared as above, or by dissolving phosphate salt for the preparation of the second aqueous solution directly into the acidic aqueous solution.

The acidic aqueous solution containing phosphate ions thus prepared is mixed with the aqueous solution of the crude extract of hyaluronic acid at the amount which can obtain the mixing ratio of the acidic aqueous solution which contains phosphate ions/the aqueous solution of the crude extract of hyaluronic acid of 0.001-100 by volume, preferably of 0.1-2 by volume, and stirred for about 10 minutes to 5 hours, preferably for 30 minutes to 1 hour. Thus prepared mixed aqueous solution of the phosphate ion-containing acidic aqueous solution and the aqueous solution of the crude extract of hyaluronic acid, is further mixed with the first aqueous solution at the amount which can obtain the molar ratio of Ca/P of 0.5-3.0 and sufficiently stirred. Alternatively, it is possible to mix the aqueous solution of the crude extract of hyaluronic acid with the first aqueous solution, and then with the phosphate ion-containing acidic aqueous solution.

The pH of the mixed aqueous solution of the phosphate ion-containing acidic aqueous solution, the first aqueous solution and the aqueous solution of the crude extract of hyaluronic acid prepared as above, is controlled to be in the range of 5-14, preferably of 8-10 with the aqueous solution of the basic compound, and stirred for 30 minutes to 24 hours, preferably for 1-3 hours.

As an alternative, before mixing the phosphate ion-containing acidic aqueous solution with the first aqueous solution, the pH of each solution may be previously controlled to be in the range of 5-14 with the aqueous solution of the basic compound, respectively. Still as another alternative, it is also possible that the phosphate ion-containing acidic aqueous solution and the first aqueous solution are mixed together, then pH of the resulted mixed solution is controlled to be in the range of 5-14 with the aqueous solution of the basic compound, and the aqueous solution of the crude extract of hyaluronic acid is added to the pH-controlled mixed solution.

Next, the specific treatment methods using calcium phosphate salt are described as follows.

In a first method using calcium phosphate salt, one or more of the calcium phosphate compounds listed in Table 2, are dissolved into the acidic aqueous solution. The concentration of the acidic aqueous solution is suitably 0.01-10M, and preferably 0.1-2M.

The acidic aqueous solution which contains calcium ions and phosphate ions prepared as above is mixed with the aqueous solution of the crude extract of hyaluronic acid at the mixing ratio of the acidic aqueous solution which contains calcium ions and phosphate ions/the aqueous solution of the crude extract of hyaluronic acid of 0.1-20 by volume, and preferably 0.5-2 by volume, and the resulted solution is sufficiently stirred for 10 minutes to 3 hours, and preferably 30 minutes to 1 hour. After sufficient stirring, the aqueous solution of the basic compound is added thereto, while stirring, so as to bring the pH in the range of 5-14, and preferably 8-10, and the resulted solution is stirred further for 30 minutes to 24 hours, and preferably for 1-3 hours to form precipitates.

In a second method using calcium phosphate salt, the aqueous solution of the basic compound is added to the acidic aqueous solution which contains calcium ions and phosphate ions to provide the pH in the range of 5-14, and preferably 8-10, and then the resulted solution is mixed with the aqueous solution of the crude extract of hyaluronic acid at the mixing ratio of the acidic aqueous solution which contains calcium ions and phosphate ions/the aqueous solution of the crude extract of hyaluronic acid of 0.1-20 by volume, and preferably of 0.5-2 by volume and stirred for 30 minutes to 24 hours, preferably for 1-3 hours.

In the step (2) of the method of purifying hyaluronic acid according to the present invention, the aqueous solution which contains precipitates resulted from the step (1) is filtered, and from the filtrate, purified hyaluronic acid is obtained as precipitates. The filtering method is not specifically limited and the filtering process is repeated until a clear solution is obtained. It may be possible to use centrifugation before filtering, and then repeat filtering until a clear solution is obtained. For removing ions potentially present in the clear solution, it is preferred to carry out ultrafiltration (UF) or dialysis.

Since the precipitate-containing aqueous solution resulted from the step (1) is a basic aqueous solution, it is preferred to make the aqueous solution to be acidic or weakly basic for effective precipitation of hyaluronic acid in the subsequent step (2). For this, to the clear solution obtained after repeated filtering, for example, an acidic aqueous solution as described above is added to bring the pH in the acidic or weakly basic range.

For precipitating hyaluronic acid from the filtrate, a solvent which very poorly dissolves hyaluronic acid as compared with the water is used and, for example, ethanol is suitably used. Additionally, water-soluble neutral salts may be added for effective precipitation of hyaluronic acid by common ion effect during the precipitation.

Hereinafter, the step (2) is further described in detail through a preferred embodiment of the present invention.

In one preferred embodiment of the step (2), the precipitate-containing aqueous solution obtained from the step (1) is centrifuged and then filtered to obtain a clear solution. To the clear solution, the acidic aqueous solution is added to bring the pH in the range of 2-10, and preferably 4.0-7.0, and the resulted solution is subjected to UF or dialysis. Into the ultrafiltrated or dialyzed solution, NaCl measured to provide the concentration of 0.3-1M, and preferably 0.5-1M is dissolved, and to the resulted solution, 95% ethanol in a volume of 1-2 times as much as the resulted solution is added so as to precipitate hyaluronic acid. The precipitated hyaluronic acid is filtered, and then the filtered hyaluronic acid is washed with 95% ethanol and then with 70-90% ethanol which has been mixed with inorganic acid, organic acid or a mixture thereof to bring the pH in the range of 1-5 by slowly stirring for 30 minutes to 5 hours, and preferably for 30 minutes to 1 hour. After completing the stirring, the precipitates are filtered, collected and subjected to freeze drying or vacuum drying and, thereby the purified hyaluronic acid can be obtained.

The method for purifying hyaluronic acid of the present invention may further comprises one or more of additional purification steps after the step (2) for obtaining hyaluronic acid with higher purity, wherein the additional purification step comprises treating the hyaluronic acid obtained from the step (2) with the calcium salt and the phosphate salt, or the calcium phosphate salt of the step (1), filtering the resultant, and precipitating hyaluronic acid from the filtrate. It is possible to obtain more highly purified hyaluronic acid by repeating the additional purification step once or more with the resulted hyaluronic acid from the step (2), and then freeze drying or vacuum drying of the resulted precipitates from the additional purification step.

The method for purifying hyaluronic acid of the present invention is applicable to the purification of hyaluronic acid originated from natural products other than rooster combs and microorganism, and further to the purification of proteins, nucleic acids or lipids.

Hereinafter, the present invention is further illustrated in more detail through the examples given below, however it should be understood that the present invention is by no means restricted thereby. Additionally, it should also be understood that, in the case of extracting hyaluronic acid from rooster combs as in the following examples, the production yield of hyaluronic acid may be greatly varied according to the size of rooster combs used.

### EXAMPLE 1

Pretreatment: A Step of Treating Rooster Combs as a Hyaluronic Acid-Containing Natural Product with Protease to Obtain a Crude Extract of Hyaluronic Acid

1 kg of rooster combs having an average square of about 5□ was thawed at 50° C. and then milled, wherein the average square was calculated by the following formula: (average width average height)÷2. The milled rooster combs were washed with 70% ethanol and then milled again. The re-milled rooster combs were washed several times with 95% ethanol. After washing, to 3000 mL of distilled water maintained at 50° C., the milled rooster combs and then protease were added and allowed to react for 24 hours. After the treatment, the separated upper layer which contained lipids was discarded and the down layer was taken to filtration. The filtered down layer was heated to 37° C. and followed by the addition of protease, and then allowed to react for 12 hours for second treatment. After the second treatment, it was filtered until a clear solution was obtained and, thereby an extract of hyaluronic acid was obtained. To the extract, NaCl measured to provide the concentration of 0.5M, was added, and hyaluronic acid was precipitated from the resulted extract in 95% ethanol. The precipitates of hyaluronic acid were separated by filtration, and subjected to freeze drying or vacuum drying to obtain the crude extract of hyaluronic acid as white powder.

(1) Step of Treating the Crude Extract Powder of Hyaluronic Acid with Calcium Salt and Phosphate Salt, or Calcium Phosphate Salt

1 g of the crude extract powder of hyaluronic acid obtained from the pretreatment step was dissolved into 1000 mL of distilled water to form 0.1% aqueous solution of the crude extract of hyaluronic acid. An aqueous solution of Ca(NO₃)₂.4H₂O was prepared to the concentration of 0.167M, and an aqueous solution of (NH₄)₂HPO₄ was prepared to the concentration of 0.1M. 500 mL of 0.167M aqueous solution of Ca(NO₃)₂.4H₂O was added to 1000 mL of 0.1% aqueous solution of the crude extract of hyaluronic acid and mixed together. To the sufficiently mixed aqueous solution, 500 mL of 0.1M aqueous solution of (NH₄)₂HPO₄, which was the same amount with the aqueous solution of Ca(NO₃)₂.4H₂O, was added and mixed together. The mixed aqueous solution of 0.1% crude extract of hyaluronic acid, Ca(NO₃)₂.4H₂O and (NH₄)₂ HPO₄ was stirred while adding NaOH, so as to control the pH to 10. After the pH control, the mixed solution was stirred for 3 hours.

(2) Step of Filtering the Resultant from the Step (1), Precipitating Hyaluronic Acid from the Filtrate and Purifying It

The mixed aqueous solution obtained from the step (1) was centrifuged until it became clear, and then was filtered. To the clear solution, HCl was added to bring the pH to 5, and the resulted solution was subjected to ultrafiltration or dialysis to obtain a clear solution again. NaCl was dissolved thereinto to provide the concentration of 0.5M, and then hyaluronic acid was precipitated from the resulted solution in 95% ethanol. The hyaluronic acid precipitates were separated by filtration, added to 70% ethanol of the pH controlled to 4, slowly stirred for 30 minutes, and then the resultant was filtered and subjected to freeze drying or vacuum drying to obtain primary purified hyaluronic acid.

The primarily purified hyaluronic acid was re-dissolved into distilled water, and the above steps of (1) and (2) was repeated once again to obtain secondary purified hyaluronic acid.

### EXAMPLE 2

The same method for purifying hyaluronic acid as in Example 1 was carried out, except that in the step (1), 250 mL of 0.1M aqueous solution of (NH₄)₂HPO₄ was added to 1000 mL of 0.1% aqueous solution of the crude extract of hyaluronic acid and mixed sufficiently, and then 0.167M aqueous solution of Ca(NO₃)₂.4H₂O was added thereto at the same amount with the 0.1M aqueous solution of (NH₄)₂HPO₄.

### EXAMPLE 3

The same method for purifying hyaluronic acid as in Example 1 was carried out except the step (1) conducted as below.

In the step (1), the crude extract powder of hyaluronic acid obtained from the pre-treatment step of Example 1 was dissolved into distilled water to prepare 1000 mL of 0.2% aqueous solution of the crude extract of hyaluronic acid. 0.85M aqueous solution of Ca(OH)₂ and 0.5M aqueous solution of H₃PO₄ were prepared, respectively. To each of the aqueous solutions of Ca(OH)₂ and H₃PO₄, an aqueous solution of NH₄OH was added to bring the pH to 11.300 mL of the pH-controlled aqueous solution of Ca(OH)₂ was added to 1000 mL of 0.2% aqueous solution of the crude extract of hyaluronic acid and mixed together, and then 300 mL, which was the same amount with the aqueous solution of Ca(OH)₂, of 0.5M aqueous solution of H₃PO₄ was further added thereto and mixed together. The mixed aqueous solution of 0.2% crude extract of hyaluronic acid, Ca(OH)₂ and H₃PO₄ was stirred for 1 hour.

### EXAMPLE 4

The same method for purifying hyaluronic acid was carried out as in Example 3, except that, in the step (1), the pH-controlled 500 mL of 0.5M aqueous solution was added to 100 mL of 0.2% aqueous solution of the crude extract of hyaluronic acid and mixed together, and thereto 0.875M aqueous solution of Ca(OH)₂ was added at the same amount with the 0.5M aqueous solution of H₃PO₄.

### EXAMPLE 5

The same method for purifying hyaluronic acid was carried out as in Example 1, except that the step (1) was carried out as below.

In the step (1), the crude extract powder of hyaluronic acid obtained from the pre-treatment step of Example 1 was dissolved into distilled water to prepare 1000 mL of 0.2% aqueous solution of the crude extract of hyaluronic acid. CaCO₃ was dissolved into 0.3M aqueous HCl solution to achieve the concentration of 17 mM, thereby preparing an aqueous HCl solution which contains calcium ions. 10 mM aqueous H₃PO₄ solution and 10 mM aqueous solution of (NH₄)₂HPO₄ were prepared respectively. 1000 mL of the prepared aqueous HCl solution which contains calcium ions were added to 1000 mL of 0.2% aqueous solution of the crude extract of hyaluronic acid and mixed, and then to the resulted solution, 1000 mL of the 10 mM aqueous H₃PO₄ solution and 1000 mL of the 10 mM aqueous solution of (NH₄)₂HPO₄ were added, and mixed together. After sufficient mixing, NH₄OH was added thereto to bring the pH to 10. After the pH control, the resulted solution was stirred for 3 hours.

### EXAMPLE 6

The same method for purifying hyaluronic acid was carried out as in Example 1, except that the step (1) was carried out as below.

In the step (1), the crude extract powder of hyaluronic acid obtained from the pre-treatment step of Example 1 was dissolved into distilled water to prepare 1000 mL of 0.2% aqueous solution of the crude extract of hyaluronic acid. 0.2M aqueous HCl solution was prepared, and thereto 2 g of octacalcium phosphate as a calcium phosphate compound was added and dissolved. To 1000 mL of aqueous solution of calcium phosphate-HCl thus prepared, 1000 mL of 0.2% aqueous solution of the crude extract of hyaluronic acid were added and stirred for sufficient mixing, during while NaOH was further added thereto so as to bring the pH to 9. After the pH control, the resulted solution was stirred for 3 hours.

### EXAMPLE 7

The same method for purifying hyaluronic acid was carried out as in Example 1, except that the step (1) was carried out as below.

In the step (1), the crude extract powder of hyaluronic acid obtained from the pre-treatment step of Example 1 was dissolved into distilled water to prepare 1000 mL of 0.3% aqueous solution of the crude extract of hyaluronic acid. 0.2M aqueous HCl solution and 1M aqueous solution of CH₃COOH were prepared respectively, and mixed at the amount which can provide the volume ratio of 0.2M HCl:1M CH₃COOH of 1:4, thereby preparing a mixed acidic aqueous solution of organic acid and inorganic acid. To the mixed acidic aqueous solution, 4 g of dicalcium phosphate anhydrate (DCPA) as a calcium phosphate compound were added and dissolved. 1000 mL of the resulted acidic aqueous solution containing calcium phosphate was added to 1000 mL of 0.3% aqueous solution of the crude extract of hyaluronic acid and stirred for sufficient mixing, during while NH₄OH was further added thereto so as to bring the pH to 10. After the pH control, the resulted solution was stirred for 1 hour.

### EXAMPLE 8

The same method for purifying hyaluronic acid was carried out as in the steps (1) and (2) of Example 1, except that a microorganism culture solution which contained hyaluronic acid was used as an aqueous solution of the crude extract of hyaluronic acid.

The following Tables 3 and 4 represent test methods for hyaluronic acid (for ophthalmic use) issued by Korea Food and Drugs Administration (KFDA), and test results of the hyaluronic acid purified by Examples 1-8 of the present invention, respectively.

TABLE 3
Test methods for hyaluronic acid (for ophthalmic use) issued by KFDA

| Content | Test methods |
|---|---|
| Hyaluronic acid | Carbazole method (Anal. Biochem. 4, 330 (1962)) |
| Proteins | Lowry's method |
| Other acidic mu- | Electrophoresis (Japanese Journal of Biochemistry 38, |
| copolysaccharides | 479 (1966)) |
| Viscosity | Brookfield III + spin LV3, 12 rpm, 25? |

TABLE 4
Purified hyaluronic acids obtained from Examples 1-8

| | Hyaluronic | | Other acidic mu- | Viscosity(0.5% |
|---|---|---|---|---|
| | acid | Proteins | copolysaccharides | aq. solution) |
| Example 1 | ≧90% | <0.02% | N.D* | ≧2400 cp |
| Example 2 | ≧90% | <0.02% | N.D. | ≧2400 cp |
| Example 3 | ≧90% | <0.02% | N.D. | ≧2400 cp |
| Example 4 | ≧90% | <0.02% | N.D. | ≧2400 cp |
| Example 5 | ≧90% | <0.02% | N.D. | ≧2400 cp |
| Example 6 | ≧90% | <0.02% | N.D. | ≧2400 cp |
| Example 7 | ≧90% | <0.02% | N.D. | ≧2400 cp |
| Example 8 | ≧90% | <0.02% | N.D. | ≧2400 cp |

*N.D: Not Detected

### INDUSTRIAL APPLICABILITY

The present invention provides a method for purifying hyaluronic acid, which can rapidly purify hyaluronic acid with high purity from a hyaluronic acid-containing extract or culture solution, and remove proteins, nucleic acids, lipids and the like in very efficient way. Moreover, the present invention overcomes the problems of the conventional purifying methods using organic solvents, simplifies the process, thereby makes the designing of suitable process for the mass production possible, and ensures high production yield. Hyaluronic acid purified by the method according to the present invention shows the purity of 90% or more, and thus it is suitable for medical use.

1. A method for purifying hyaluronic acid comprising the steps of: (1) treating a crude extract of hyaluronic acid with calcium salt and phosphate salt, or calcium phosphate salt, wherein the crude extract is obtained by treating natural product containing hyaluronic acid with protease, or from a culture solution containing hyaluronic acid originated from microorganism; and (2) purifying the hyaluronic acid by filtering the resultant from the step (1) and precipitating the hyaluronic acid from the filtrate.

2. The method for purifying hyaluronic acid according to claim 1, characterized in that the crude extract of hyaluronic acid is obtained by treating rooster combs as the natural product containing hyaluronic acid with protease at 30-60° C. for 3-72 hours, and separating and removing lipid phase.

3. The method for purifying hyaluronic acid according to claim 1, characterized in that the calcium salt is at least one selected from the group consisting Ca(OH)₂, CaCO₃, Ca(NO₃)₂.4H₂O, Ca(CH₃COO)₂.H₂O, CaCl₂ and CaSO₄.2H₂O.

4. The method for purifying hyaluronic acid according to claim 1, characterized in that the phosphate salt is at least one selected from the group consisting of H₃PO₄, (NH₄)₂HPO₄, H₄P₂O₇, NaH₂PO₄ and KH₂PO₄.

5. The method for purifying hyaluronic acid according to claim 1, characterized in that the calcium salt and the phosphate salt are used at the molar ratio of Ca/P of 0.5-3.0.

6. The method for purifying hyaluronic acid according to claim 1, characterized in that the calcium phosphate salt is at least one selected from the group consisting of tetracalcium phosphate, hydroxyapatite, nonstoichiometric hydroxyapatite, amorphous calcium phosphate, tricalcium phosphate, octacalcium phosphate, dicalcium phosphate dihydrate, dicalcium phosphate anhydrate, calcium pyrophosphate, calcium pyrophosphate dihydrate, heptacalcium phosphate, tetracalcium dihydrogen phosphate, monocalcium phosphate monohydrate and calcium metaphosphate.

7. The method for purifying hyaluronic acid according to claim 1, characterized in that the crude extract of hyaluronic acid in the step (1) is treated in the form of aqueous solution, and the calcium salt and the phosphate salt, or the calcium phosphate salt is mixed, in the form of aqueous solution, with the aqueous solution of the crude extract of hyaluronic acid.

8. The method for purifying hyaluronic acid according to claim 7, characterized in that at least one of the aqueous solutions of the calcium salt, the phosphate salt, and the calcium phosphate salt as an acidic aqueous solution, is mixed with the aqueous solution of the crude extract of hyaluronic acid.

9. The method for purifying hyaluronic acid according to claim 8, characterized in that the acidic aqueous solution comprises at least one acid selected from the group consisting of HCl, HNO₃, HF, H₂SO₄, HClO₄, HClO₃, H₂CO₃, CH₃COOH, C₂H₂(COOH)₂, HOOCCH₂CH₂COOH, CHCOCOOH and CH₃(CH₂)₁₄COOH.

10. The method for purifying hyaluronic acid according to claim 7 or 8, characterized in that the aqueous solution of the crude extract of hyaluronic acid is treated under the condition of pH 5-14, simultaneously with mixing or after mixing.

11. The method for purifying hyaluronic acid according to claim 10, characterized in that the condition of pH 5-14 is achieved by using basic compound.

12. The method for purifying hyaluronic acid according to claim 11, characterized in that the basic compound is at least one selected from the group consisting of NaOH, KOH, LiOH, NH₄OH, Mg(OH)₂, trishydroxymethyl-aminomethane, bis-2-hydroxyethyl-aminotris-hydroxymethyl-methane and 1,3-bis-[tris-hydroxymethyl]-methyl-aminopropane.

13. The method for purifying hyaluronic acid according to claim 1, characterized in that, in the step (1), the aqueous solution of the crude extract of hyaluronic acid is treated with, as a source of calcium salt, an aqueous solution (first aqueous solution) which does not contain phosphate ions but calcium ions, and as a source of phosphate salt, an aqueous solution (second aqueous solution) which does not contain calcium ions but phosphate ions.

14. The method for purifying hyaluronic acid according to claim 13, characterized in that, the calcium ion concentration of the first aqueous solution and the phosphate ion concentration of the second aqueous solution are 0.001-10M, and the molar ratio of the calcium salt to the phosphate salt, Ca/P, used is 0.5-3.0.

15. The method for purifying hyaluronic acid according to claim 13, characterized in that at least one of the first and the second aqueous solution is an acidic aqueous solution which comprises at least one acid selected from HCl, HNO₃, HF, H₂SO₄, HClO₄, HClO, H₂CO₃, CH₃COOH, C₂H₂(COOH)₂, HOOCCH₂CH₂COOH, CHCOCOOH and CH₃(CH₂)₁₄COOH.

16. The method for purifying hyaluronic acid according to claim 13 or 14, characterized in that the aqueous solution of the crude extract of hyaluronic acid is treated by forming a mixed aqueous solution of the first and the second aqueous solution and the aqueous solution of the crude extract of hyaluronic acid, through a first mixing method in which the second aqueous solution is added to the mixed aqueous solution of the first aqueous solution and the aqueous solution of the crude extract of hyaluronic acid, or
a second mixing method in which the first aqueous solution is added to the mixed aqueous solution of the second aqueous solution and the aqueous solution of the crude extract of hyaluronic acid, or
a third mixing method in which the aqueous solution of the crude extract of hyaluronic acid is added to each of the first and the second aqueous solution, and then each of the resulted solutions are mixed together, or
a fourth mixing method in which the aqueous solution of the crude extract of hyaluronic acid is added to the mixed aqueous solution of the first and the second aqueous solution.

17. The method for purifying hyaluronic acid according to claim 16, characterized in that at least one aqueous solution among
the first aqueous solution;
the second aqueous solution;
the mixed aqueous solution of the first aqueous solution and the aqueous solution of the crude extract of hyaluronic acid;
the mixed aqueous solution of the second aqueous solution and the aqueous solution of the crude extract of hyaluronic acid;
the mixed aqueous solution of the first and the second aqueous solution; and
the mixed aqueous solution of the first and the second aqueous solution and the aqueous solution of the crude extract of hyaluronic acid is controlled to have the condition of pH 5-14.

18. The method for purifying hyaluronic acid according to claim 17, characterized in that at least one basic compound selected from the group consisting of NaOH, KOH, LiOH, NH₄OH, Mg(OH)₂, trishydroxymethyl-aminomethane, bis-2-hydroxyethyl-aminotris-hydroxymethyl-methane and 1,3-bis-[tris-hydroxymethyl]-methyl-aminopropane, is used to control the pH in the range of 5-14.

19. The method for purifying hyaluronic acid according to claim 16, characterized in that
the mixing ratio of the first aqueous solution to the aqueous solution of the crude extract of hyaluronic acid by volume (the first aqueous solution/the aqueous solution of the crude extract of hyaluronic acid by volume) in the first or the third mixing method,
the mixing ratio of the second aqueous solution to the aqueous solution of the crude extract of hyaluronic acid by volume (the second aqueous solution/the aqueous solution of the crude extract of hyaluronic acid by volume) in the second or the third mixing method, and
the mixing ratio of the mixed aqueous solution of the first and the second aqueous solution to the aqueous solution of the crude extract of hyaluronic acid by volume (the mixed aqueous solution of the first and the second aqueous solution/the aqueous solution of the crude extract of hyaluronic acid by volume) in the fourth mixing method are 0.001-100.

20. The method for purifying hyaluronic acid according to claim 1, characterized in that, in the step (1), the aqueous solution of the crude extract of hyaluronic acid is treated with an aqueous solution of calcium phosphate salt by mixing with the aqueous solution of at least one calcium phosphate salt selected from the group consisting of tetracalcium phosphate, hydroxyapatite, nonstoichiometric hydroxyapatite, amorphous calcium phosphate, tricalcium phosphate, octacalcium phosphate, dicalcium phosphate dihydrate, dicalcium phosphate anhydrate, calcium pyrophosphate, calcium pyrophosphate dihydrate, heptacalcium phosphate, tetracalcium dihydrogen phosphate, monocalcium phosphate monohydrate and calcium metaphosphate.

21. The method for purifying hyaluronic acid according to claim 20, characterized in that, the aqueous solution of calcium phosphate salt is an acidic solution which comprises at least one acid selected from HCl, HNO₃, HF, H₂SO₄, HClO₄, HClO, H₂CO₃, CH₃COOH, C₂H₂(COOH)₂, HOOCCH₂CH₂COOH, CHCOCOOH and CH₃(CH₂)₁₄COOH.

22. The method for purifying hyaluronic acid according to claim 20 or 21, characterized in that the aqueous solution of the crude extract of hyaluronic acid is treated under the condition of pH 5-14, simultaneously in or after mixing with the aqueous solution of calcium phosphate salt.

23. The method for purifying hyaluronic acid according to claim 22, characterized in that, the condition of pH 5-14 is achieved by using at least one basic compound selected from the group consisting of NaOH, KOH, LiOH, NH₄OH, Mg(OH)₂, trishydroxymethyl-aminomethane, bis-2-hydroxyethyl-aminotris-hydroxymethyl-methane and 1,3-bis-[tris-hydroxymethyl]-methyl-aminopropane.

24. The method for purifying hyaluronic acid according to claim 20, characterized in that the mixing ratio of the aqueous solution of calcium phosphate salt to the aqueous solution of the crude extract of hyaluronic acid by volume (the aqueous solution of calcium phosphate salt/the aqueous solution of the crude extract of hyaluronic acid by volume) is 0.1-20.

25. The method for purifying hyaluronic acid according to claim 1, characterized in that, in the step (2), the resultant from the step (1) is centrifuged until a clear solution is obtained, and then the clear solution is filtered.

26. The method for purifying hyaluronic acid according to claim 25, characterized in that, an acidic aqueous solution comprising inorganic acid, organic acid or the mixture thereof is added to the clear solution obtained from the centrifugation and filtration, to control the pH in the range of 2-10.

27. The method for purifying hyaluronic acid according to claim 1, characterized in that, in the step (2), after filtering the resultant from the step (1), the hyaluronic acid is precipitated therefrom by using ethanol.

28. The method for purifying hyaluronic acid according to claim 27, characterized in that water-soluble neutral salt is added to the solution where the precipitation of hyaluronic acid is occurred.

29. The method for purifying hyaluronic acid according to claim 1, characterized in that, in the step (2), after filtering the resultant from the step (1) and precipitating hyaluronic acid, the precipitated hyaluronic acid is washed with ethanol, and then washed again with 70-90% ethanol of which pH is controlled in the range of 1-5.

30. The method for purifying hyaluronic acid according to claim 1, characterized by further comprising one or more of additional purification steps after the step (2), wherein the additional purification step comprises treating the hyaluronic acid obtained from the step (2) with the calcium salt and the phosphate salt, or the calcium phosphate salt of the step (1), filtering the resultant, and precipitating hyaluronic acid from the filtrate.

31. The method for purifying hyaluronic acid according to claim 1, characterized in that the crude extract of hyaluronic acid in the step (1) is treated with calcium salt and phosphate salt, or calcium phosphate salt at the temperature of 4-90° C.
